(19) United States
(12) Patent Application Publication
(10) Pub. No.: US 2008/0292598 A1
(43) Pub. Date: Nov. 27, 2008

(54) CHIMERIC TRANSPLANT

(76) Inventor: HARRIS F. BROTMAN, La Jolla, CA (US)

Correspondence Address: STEMEDICA CELL TECHNOLOGIES, INC, 5375 MIRA SORRENTO PLACE, SUITE 100, SAN DIEGO, CA, 92121, US

(21) Appl. No.: 12/110,227

(22) Filed: Apr. 25, 2008

Related U.S. Application Data
(60) Provisional application No. 60/914,122, filed on Apr. 26, 2007.

(51) Int. Cl. A61K 35/50; C12Q 1/02; A61P 43/00

(52) U.S. Cl. 424/93.7; 435/29

(57) ABSTRACT

Compositions comprising amniotic fluid stem cells which are derived from non-identical donor sources. Donors may be non-identical siblings, non-identical twins, and/or donors which are unrelated by a familial relationship. Also disclosed are methods for making such amniotic stem cell compositions, and methods for their use, such as therapeutic stem cell transplantation.

This application claims priority to provisional application Ser. No. 60/914,122 filed Apr. 26, 2007.

### FIELD OF THE INVENTION

The present invention is directed to compositions of amniotic fluid cells and stem cell derivatives thereof. Included are methods of making the compositions, preparing transplants therefrom, and administering such transplants to recipients in the treatment of diseased or lesioned tissues.

### BACKGROUND

Human amniotic fluid has been used in prenatal diagnosis for more than 70 years. It has proven to be a safe, reliable, and simple screening tool for a wide variety of developmental and genetic diseases. A number of groups have isolated subsets of cells found in amniotic fluid and placenta and found them to be capable of prolonged undifferentiated proliferation as well as able to differentiate into multiple tissue types encompassing the three germ layers.

Amniotic fluid is known to contain multiple cell types derived from the developing fetus. Cells within this heterogeneous population can give rise to diverse differentiated cells including those of adipose, muscle, bone and neuronal lineages. Clonal human amniotic fetal cells (AFS) cells meet a commonly accepted criterion for pluripotent stem cells, which is that they give rise to adipogenic, osteogenic, myogenic, endothelial, neurogenic and hepatic lineages, inclusive of all embryonic, germ layers.

AFS cells are expected to open up novel cell-based therapeutic applications. Amniotic fluid cells, and the progenitor and stem cells derived therefrom, are widely investigated for replacing dysfunctional cells within a tissue. AFS cells can yield differentiated cells that express lineage-specific markers and acquire characteristic functions in vitro. Initial studies, indicate that AFS cells induced toward particular lineages can generate specialized cells after implantation in vivo. AFS cells directed to neural lineage differentiation by exposure to nerve growth factor (NGF) are able to widely engraft the developing mouse brain in a manner similar to that previously observed for neural stem cells.

Viable adherent cells from amniotic fluid may be classified into three major groups based on their morphological, biochemical and growth characteristics: epithelioid (E-type), amniotic fluid (AF-type) and fibroblastic (F-type) cells. Human amniotic fluid stem cells can be isolated and manipulated as cell lines. AFS cells have the potential to treat or cure a myriad of diseases, including Parkinson's and Alzheimer's diseases, heart disease, diabetes, stroke, spinal cord injuries, and burns. Subpopulations of fetal mesenchymal cells can be consistently isolated from human amniotic fluid and rapidly expanded in vitro. The amniotic fluid can be a valuable and practical cell source for fetal tissue engineering. The lineages of these amniotic subpopulations of amniocytes have been determined by immunofluorescent staining with antibodies against markers, expressed by stem cell lineages through development.

Bossolasco, P. (Cell Research (2006)16: 329-336) extensively characterized unselected amniotic cells and tested their multilineage differentiation capacity in vitro. Interestingly AFCs were positive for markers characteristic of both immature, lineage-committed cells and mature/functional phenotype cells. AFCs also showed a differentiation potential towards different lineages. Karlmark, K. R. et al. International Journal Of Molecular Medicine 16: 987-992, 2005 reports the first/isolation of genetically engineered human amniotic fluid cells and describe a protocol for transfecting these cells.

### SUMMARY OF THE INVENTION

The invention involves a composition which comprises amniotic fluid cells or derivatives of those cells. The composition has a mixed genotype, formed by seeding cells of a genotype with at least a second genotype of cells from another donor, i.e. the donors are non-identical. The non-identical donors are siblings or non-siblings. Sibling donors, in one embodiment, are non-identical twins.

An aspect of the invention is directed to methods of culturing pure populations of human amniotic stem cells of one genotype with at least amniotic stem cells of another genotype. A method of creating a chimeric composition derived from at least two non-identical donors involves obtaining a first culture of amniotic fluid cells of one genotype from a first donor; obtaining a second culture of amniotic fluid cells of another genotype from another donor; and co-culturing the first culture and the second culture such that a chimeric composition is formed.

A further aspect involves methods of fabricating a transplant which involves a step of combining amniotic fluid cells of one genotype with at least amniotic fluid cells of another genotype. Coculturing of the genotypes produces a chimeric, composition from which a chimeric transplant is formed.

Other aspects of the invention are directed to methods of treating an individual in need of a transplant. The method comprising the step of administering to an individual a chimeric transplant. A transplant of the invention comprises a stem cell composition suitable for administration to a human recipient. The transplant comprises a therapeutically effective combination of stem cells derived from at least two nonidentical donors. The administration of the transplant induces regeneration of tissue in a recipient.

Another dimension of the invention involves a competitive repopulation assay for measuring the relative contribution of each genotype of donor cell in a mixed genotype transplant formed from a mixed amniotic stem cell composition. The assay involves tracing the distribution of cells having a distinguishable marker in one or more tissues of the recipient. By quantitating the donor cell types in one or more tissues of the recipient, the method determines the relative ability of each donor to populate one or more tissues in a recipient.

The invention also features an assay for analyzing an effect of a candidate compound on a cellular coculture comprising a mixed composition.

### DETAILED DESCRIPTION OF THE INVENTION

The present, invention is based upon a mixed composition of amniotic fluid cells, or derivatives thereof, which have been obtained from at least two non-identical donors. A mixed population of stem cells which have pluripotent differentiation capacity (and therefore are a viable) is derived from the mixed composition of amniotic fluid cells from non-identical donors. The mixed compositions of the invention are used to provide a source of stem cells that can be used therapeutically. Mixed cell compositions obtained from non-identical donors of chorionic villus and placenta are another aspect of the invention.

### DEFINITIONS

Stem cells are undifferentiated cells defined by the ability of a single cell to both self-renew, and differentiate into progeny cells, including self-renewing progenitors, non-renewing progenitors, and terminally differentiated cells. Stem cells are also characterized by their ability to differentiate in vitro into functional cells of various cell lineages. Stem cells may have varying degrees of potency. Pluripotent stem cells are capable of giving rise to cells belonging to each of the three embryonic germ layers (i.e. the endoderm, mesoderm and ectoderm). The differentiation versatility of pluripotent” or “pluripotential” cells extends to a capacity to differentiate into at least neurogenic phenotype, osteogenic phenotype, hematopoietic phenotype, adipogenic phenotype, myogenic phenotype, hepatic phenotype and endothelial phenotype in appropriate inducing conditions. Multipotent stem cells are more lineage restricted than pluripotent stem cells as they are only capable of forming cells from a single lineage (e.g. ectodermal cells). Stem cells may also be progenitor cells (i.e. precursor cells) which are lineage-committed cells capable of both dividing and differentiating into a specific terminal cell type.

Stem cells are also categorized on the basis of their source. An adult stem cell is generally a pluripotent or multipotent undifferentiated cell found in tissue comprising multiple differentiated cell types. The adult stem cell can renew itself. Under normal circumstances, it can also differentiate to yield the specialized cell types of the tissue from which it originated, and possibly other tissue types.

A fetal stem cell is, one that originates from fetal tissues or membranes. Amniotic fluid stem cells are one example of a fetal stem cell.

Preferably the pluripotent cell has the capacity to differentiate to any of the mammalian body's about 260 different cell types

As used herein, the term “multi-potent” refers to the ability of stem cells to differentiate into at least two cell types belonging to the same germ lineage (e.g. at least two cell types belonging to the mesodermal germ lineage).

In certain embodiments of the invention, the stem cells are human stem cells that can be propagated for an indefinite period of time in an undifferentiated state. The term “undifferentiated” refers to cells that have not become specialized cell types. Typically, the cells are grown in a nutrient medium. A “nutrient medium” is a medium for culturing cells containing nutrients that promote proliferation. The nutrient medium may contain any of the following in an appropriate combination: isotonic saline, buffer, amino acids, antibiotics, serum or serum replacement, and exogenously added facturs.

The phenotype of a subject cell, cell line, culture, coculture, or transplant, is the collection of all its “manifested attributes.” The phenotype of a subject cell, cell line, culture, coculture, or transplant refers to the outward, observable (behavioral or physiological) expression of the genotype(s) therein. The phenotype is the joint product of the genotype and the environment. The phenotype also refers to the effects of a subject cell, cell line, or transplant as manifest in an environment, or as manifest in a transplant recipient. Phenotype refers to characteristics associated with a particular genotype at multiple levels of description, from the molecular to the cellular, systems, cognitive and behavioral levels.

Cell differentiation means progressive restriction of the developmental potential, and increasing specialization of function, that leads to the formation of specialized cells, tissues, and organs.

Various terms are used to describe cells in culture. “Cell culture” includes, but is not limited to, cells taken from a living organism and grown under controlled conditions (“in culture” or “cultured”). A “primary cell culture” is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number. This is referred to as “doubling time.”

As used herein “coculture” means a technique of culturing a mixture of cell genotypes (i.e. mixed cell types in vitro) to allow their synergistic or antagonistic interactions to manifest, such as on cell differentiation. Coculture can involve different types of cells, tissues, or organs from normal or disease states. Coculture can further involve the culturing together of two or more genetically distinct cell populations, wherein the cell populations belong to a single cell type (e.g. a heterogenous population of neural progenitors).

A stem cell “line” is the family of cells derived from a single original stem cell. A stem cell line may also be referred to as a “clone,” “clonal line,” or “clonal population” of cells.

The terms “derive,” “derived from” and “derivative,” as used to refer to a cell indicates that the cell came from a specific source such as, for example, a tissue, a clonal cell line, a body fluid (e.g. amniotic fluid), or a primary cell culture. For example, when a cell population is expanded from a clonal cell that was isolated from amniotic fluid cells, the cell population may be described as being derived from amniotic fluid cells and/or a clonal cell isolated from amniotic fluid cells.

The term “isolated,” or “purified” refers to a cell which has been separated, from its natural environment. This term includes gross physical separation from its natural environment and alteration of the cell's relationship with the neighboring cells with which it is in direct contact by, for example, dissociation. When used to refer to a population of cells, the term “isolated” includes populations of cells which result from proliferation of the isolated cells of the invention. “Isolated” also refers to cells or populations of cells isolated from a tissue preparation through such procedures as affinity chromatography and FACS. Populations of isolated cells are about 30%, 35%, 40%, 45%, 50%, 55%, 60%, 65%, 70%, 75%, 80%, 85%, 90%, 95% or 99% free from other undesired, contaminating cells. In another embodiment, the isolated stem cells also are substantially free of soluble, naturally occurring molecules.

The lineage of a cell relates to its derivation from less differentiated cells or tissue. Cells committed to differentiate or give rise to a defined range of differentiated cells, in principle, represent the ancestry (lineage) of an adult cell. Differentiated cells are thus derived from an ancestry of cell(s) committed to give rise to them.

As used herein, the term “chimera” refers to an organism that consists of cells of different genotype, each genotype derived from a genetically distinct second organism.

In the context of the present invention, “chimera” also refers to admixed cell culture or a transplant comprising a mixed cell culture. Accordingly, a chimera's cells derive from two or more distinct zygote lineages. Chimera also denotes cell populations ex vivo which are mixtures of genotypes, i.e. produced by combining in culture cells of different genetic origins. A transplant chimera is formed from aggregates of genetically different groups of cells derived from the same or different species. A transplantation chimera is an organism that, as a result of transplantation of donor tissue or cells, consists of two or more cell lines descended from at least two zygotes. A transplant recipient in which a monogenomic transplant engrafts is a chimera, whereas the recipient would be a double chimera if the transplant itself was a chimera.

A chimera made from cell or tissue components derived from two different genotypes has the equivalent of two genomes, or two genotypes.

“Mixed” as used herein refers to compositions of cells (e.g. suspensions or adherent arrangements, otherwise cells cultured ex vivo, or in a form suitable for transplanting or grafting to a host or recipient) which are combinations of cells of different genotypes.

The phenotype (behavior) of a chimera originates from the integration of genetically distinct cells. In the case of a chimeric graft, the graft expresses a chimeric phenotype.

As used herein, “clone” means a cell line in which each cell is identical to all the others because they are all derived from cell division from a common ancestor. A “clone,” or “clonal cell,” is a line of cells that is genetically identical to the originating cell. This cloned line is produced by cell division (mitosis) of the originating cell. The term “clonal population” in reference to the cells of the invention shall mean a population of cells that is derived from a clone. A cell line may be derived from a clone and is an example of a clonal population.

The term “cell line” refers to a population of cells cultured in vitro that has descended through one or more generations (and possibly cultures) from a single primary culture or a clone. The cells of a cell line share common characteristics.

Biclonality is a condition in which some cells have markers of one cell line of a first genotype, and other cells have markers of another cell line of a second genotype. Each cell line is derived from a different zygote. Biclonality is the simplest form of polyclonality.

The term “genotype” refers to the alleles present in aft individual cell at the locus (loci) under consideration. Alternatively, a genotype refers to the sum of all the alleles present in a genome.

A genome is the complete DNA sequence of an organism.

A sibling or sib refers to a brother of sister.

A sibship means all the brothers and sisters in one family.

As used herein, a “mosaic” is an organism or culture or aggregate that consists of cells of more than one genotype, i.e. a mixed genotype. Chimeras are mosaics under this definition.

Amniocytes are fetal cells obtained by amniocentesis from amniotic fluid. Cultured and uncultured amniocytes have been used extensively for prenatal diagnosis.

A monomorphic cell population refers to a population consisting of cells all of the same genotype.

“Regenerative” refers to the ability of a substance to restore, supplement or otherwise rehabilitate the natural function of a tissue. This ability may be conferred by, for example, treating a dysfunctional tissue with regenerative cells. Regenerative cells treat dysfunctional tissue by replacing it with new cells capable of performing the tissue's natural function, or by helping to restore the natural activity of the dysfunctional tissue.

The terms “restore,” “restoration” and “correct” are used interchangeably herein and refer to the regrowth, augmentation, supplementation, and/or replacement of a defective tissue with a new and preferentially functional tissue. The terms include the complete and partial restoration of a defective tissue. Defective tissue is completely replaced if it is no longer present following the administration of the inventive composition. Partial restoration exists where defective tissue remains after the inventive composition is administered. The terms “restore,” “restoration” and “correct” also refer to an improvement in the natural function of a defective tissue, with or without replacing the defective tissue.

The phrase “effective amount” refers to a concentration or amount of a reagent or pharmaceutical composition (e.g. growth factor, differentiation agent, trophic factor, cell population or other agent), that is effective for producing an intended result, including cell growth and/or differentiation in vitro or in vivo, or a regenerative or restorative effect. With respect to the administration of one or more populations of regenerative cells as disclosed herein, an effective amount may range from as few as several hundred or fewer cells, to as many as several million cells or more. It will be appreciated that the number of cells to be administered will vary depending on the particular needs that a targeted disorder requires.

The terms “specific binding,” or “specifically binding,” as used herein, refers to the interaction between an antibody and a cell marker (e.g. c-kit) or a fragment thereof, including the markers expressed by cells present in a chorionic villus, amniotic fluid or a placenta sample. The interaction is dependent upon the presence of a particular structure, i.e., the antigenic determinant or epitope of a cell marker recognized by the binding molecule (i.e. an antibody) that specifically binds the marker. For example, in the presence of an antibody that is specific for c-kit epitope “A,” labeled and unlabled epitope A proteins will compete with one another for antibody binding sites such that the amount of labeled epitope A bound to the antibody will be reduced.

### Methods

The present invention relies on technologies well known in the art for isolation and expansion of undifferentiated and differentiated cells from amniotic fluids for morphological characterization of cell types, for FAGS analysis of multipotent/pluripotent/progenitor and differentiated cells, for stem cell markers expressed by multipotent/pluripotent/progenitor and differentiated cells, and for cell culture induction methods for differentiating amniotic fetal cells into cells of ectoderm, mesoderm and endoderm. In addition to the differentiation paths mentioned below, pluripotent AFS cells are capable of other multipotent differentiation pathways. Differentiation methods are known in the art and can be found, for example, in U.S. Pat. No. 5,827,740; Yoo J U, et al., Jour. Bone Joint Surg. (1998) 80-A(12):1745-1757; Jaiswal N, et al., Journal of Cellular Biochem (1997) 64:295-312; Tsai M-S, et al., Human Reproduction (2004) 19(6):1450-1456; Kogler G, et al; J. Experimental Med (2004) 200(2): 123-135; and Lodie T A et al, Tissue Eng (2002) 8(5):739-753; each of which is incorporated by reference herein in its entirety.

Methods well known in the art are available for obtaining human fetal cells from amniotic fluid at 12 weeks to term, such method including, but not limited to amniocentesis and the sampling of fetal tissue froth chorionic villus (see e.g. US Published Application 2005 0124 003 and http://www.icongrouponline.com/Health/Amniocentesis_Ph.html which are incorporated by reference).

Methods for isolating amniotic stem cells lines with potential for therapy are also disclosed in De Coppi, P. et al., Isolation of Amniotic Stem Cell Lines With Potential for Therapy, 2007, Nature Biotechnology 25(1): 100-106 (incorporated herein by reference). These methods include: isolation of AFS cells; telomere length assay; retroviral marking-differentiation of amniotic fetal cells in culture; differentiation of cell lineages including adipogenic, osteotgenic, myogenic, endothelial, neurogenic, hepatic; analysis of mRNA by RC-PCR; nestin immunofluorescence; whole cell voltage clamp recording; l-glutamate secretion; amniotic fetal cell imolantion in mouse brain; urea production; and printed scaffolds for bone differentiation, implantation of printed bone construct. (ibid)

U.S. Published Application No. 2005/0054093 discloses Multipotent Amniotic Fetal Stem Cells (MAFSC), which are immortal in culture, maintain euploidy for more than 1 year in culture, share markers with human embryonic stem cells, and are capable of differentiating into all three germ layers of the developing embryo (i.e. endoderm, mesoderm and ectoderm). These human stem cells are found in the amnion harvested during the second trimester of human pregnancies. A variety of monoclonal MAFSCs with phenotypic-variations isolated from amniotic fluid are taught U.S. Published Application No. 2005/0054093.

Additionally available to workers in this field of cell-based therapeutics/regeneration therapy based on amniotic fluid cells are the following references which deal with molecular biology, microbiology, animal cells, cell lines, propagation processes, maintaining or preserving an animal cell or composition thereof, processes for isolating or separating an animal cell or composition thereof, processes for preparing a composition containing an animal cell, and culture media therefore, all of which are incorporated by reference: U.S. Published Application Nos; 2005/0054093 and 2005/0042595; (Bossolasco P et al.) Molecular and phenotypic characterization of human amniotic fluid cells and their differentiation potential (Cell Research (2006)16: 329-336); P. Dc Coppi, G. Schuch, A. Atala, Fetal cell culture in Methods of Tissue Engineering, A. Atala, R. P. Lanza, R. P. 2002 (San Diego: Academic Press. Cap. 71 p. 875); Delo, D. M., et al., Amniotic fluid and placental stem cells, Methods Enzymol (2006) 419:426-438; J. Kim et al. Human amniotic fluid-derived stem cells have characteristics of multipotent stem cells; Sakuragawa N, Thangavel R, Mizuguchi M, Hirasawa M, Kamo I. Expression of markers for both neuronal and glial cells in human amniotic epithelial cells. Neurosci Lett 1996; 209:9-12; Karlmark et al. Activation of ectopic Oct-4 and, Rex-1 promoters in human amniotic fluid cells, International Journal Of Molecular Medicine 16: 987-992, 2005; Ming-Song Tsai, et al. Clonal Amniotic Fluid-Derived Stem Cells Express Characteristics of Both Mesenchymal and Neural Stem Cells, Human Reproduction, Vol. 19, No, 6, 1450-1456, June 2004; Pieternella S. in H Anker, et al. Blood, 15:1548-1549; U.S. Published Application 2005 0118 712—Two-stage culture protocol for isolating mesenchymal stem cells from amniotic fluid; 178 Prusa A-R et al., “Oct-4-expressing cells in human amniotic fluid: a new source for stem cell research?”, Human Reproduction 18, 1489-1493; 2003; Pfusa, Andrea-Romana, Amniotic fluid cells and human stem cell research—a new connection, Med Scio Monit, (2002)8(11): RA2530257; Amnion-Derived Stem Cells, Black I. et al. UMDNJ-Rober Wood Johnson Medical School; A Real-Time PGR Approach to Evaluate Adipogenic Potential of Amniotic Fluid-Derived Human Mesenchymal Stem Cells. P D Gemmis. C Lapucci, M Bertelli, A Tognetto, E Fanin, R Vettor, C Pagano, M Pandolfo. A Fabbri, BIRD Europe Institute, Vicenza, Italy; and U.S. Pat. No. 6,790,614 drawn to selectable cell surface marker genes.

Immunostaining of cells is achieved with methods described in U.S. Published Application No. 2005/0124003, or by other immunostaining techniques well known in the art. Any immunoassay for detecting the presence of markers on whole cells, using marker specific antibodies, may be performed on cell lysates, fixed intact cells, frozen sections, and the like.

The use of cell surface antigens on amniotic fluid cells and cell derivatives thereof provides a means for the positive immunoselection of fetal stem cell populations, as well as for the phenotypic analysis of progenitor cell populations using, for example, flow cytometry. Cells selected for expression of an antigen [i.e. cell marker(s)] may be further purified by selection for other stem cell and progenitor cell markers, including, but not limited to, human embryonic stem stage specific markers. Alternatively, for the preparation of substantially pure pluripotent fetal stem cells, a subset of stem cells can be separated from other cells on the basis of antibody specific-binding properties and the resulting marker positive stem cells may be further separated by binding to other surface markers known in the art (U.S. Published Application No. 2005/0124003). Any technique may be employed which is not unduly detrimental to the viability of the selected cells.

Instructions for separating cell populations based on marker selection are well known in the art. Essentially, an antibody specific for a target marker is added to a cell sample. The amount of specific antibody necessary to bind a particular, eel), subset is empirically determined by performing a test separation and analysis. Cell specific antibodies are incubated for a period of time sufficient for complexes to form, usually at least about 5 minutes, more usually at least about 1.0 minutes, and usually not more than one hour, more usually not more than about 30 minutes.

### Selection and Enrichment

The existence of stem cells is inferred by the analysis of their differentiated progeny, using a complex series of cloning and differentiation assays. There are no markers which specifically identify all stem cells. Stem cells are defined by their immunophenotypic profile, their characteristic morphology, and by their extensive capacity for self-renewal while retaining their ability to differentiate into a number of tissue-type lineages.

Numerous methods are known in the art for selecting or enriching populations of amniotic cells for pluripotential cells. One of skill in the art can derive a population of cells by immunoselection using a species of antibody or set of antibody species that specifically bind target cell markers. For example, U.S. Published Application November 2005/0124003, incorporated herein by reference, teaches that a preferred set of amniotic fluid cells is c-kit positive. Thus, the c-kit marker can be used to isolate these cells.

By way of example and without limiting the methods of the invention, detailed protocols of c-kit selection are disclosed in U.S. Published Application No. 2005/0124003. Antibodies reactive with the c-kit or portions thereof can be used to isolate e-kit positive cells. Such antibodies specifically bind with the c-kit protein or a portion thereof. The antibodies can be polyclonal or monoclonal, and the term antibody is intended to encompass polyclonal and monoclonal antibodies, and functional fragments thereof. The terms polyclonal and monoclonal refer to the degree of homogeneity of an antibody preparation, and are not intended to be limited to particular methods of production. Examples of commercially available useful c-kit antibodies are disclosed in U.S. Published Application No. 2005/0124003.

Additional examples of commercially available antibodies include, but are not limited to, YB5.B8 monoclonal antibody which is specific for human CD117 (eBioscience, San Diego, Calif.), an antibody produced against a human leucaemic cell line UT7 transfected with CD117 cDNA (Chemicon International, Temecula, Calif.), a polyclonal antibody produced against the C-terminal end of CD117 (Assay Designs Inc., Ann Arbor, Mich., catalog No. 90572), and clone 28 c-kit monoclonal antibody (catalog no. 612318, from BD). Further, antibodies recognizing c-kit or fragments thereof may be obtained or prepared as discussed in U.S. Pat. No. 5,454,533, incorporated herein by reference.

The number of designated marker(s)—positive cells in a cell population can be determined in any well known method known to one skilled in the art such as, for example, FACS analysis. Alternatively, magnetic cell sorting technology (MACS) can be used to separate cells (see, e.g. Miltenyi Biotech, Inc., Auburn, Calif.). In MACS, the marker positive cells of choice can be separated from the mixture of chorionic villus cells, amniotic fluid, and placenta cells to very high purity.

The cells may additionally be incubated with antibodies, or binding molecules specific for cell surface markers known to be present or absent on the fetal stem cells. For example, cells expressing SSAE1 marker can be negatively selected for.

The labeled cells are separated in accordance with the specific antibody preparation. Fluorochrome labeled antibodies are useful for FACS separation, magnetic particles for immunomagnetic selection, particularly high gradient magnetic selection (HGMS), etc. Exemplary magnetic separation devices are described in WO 9007380, PCT U59600953, and EP 438,520.

The purified cell population may be collected in any appropriate medium (U.S. Published Application No. 2005/0124003).

Populations highly enriched for pluripotent fetal stem cells are achieved in this manner. In some instances, the desired cells will be 30% or more of the cell composition, 50% or more of the cell population, or 90% or more of the cell population, or even 95% or more (substantially pure) of the cell population.

Pluripotent or multipotent AFS cells may be grown in an undifferentiated state for as long as desired (and optionally stored as described herein), and can then be cultured under certain conditions to allow progression to a differentiated state. By “differentiation” is meant the process whereby an unspecialized cell acquires the features of a specialized cell such as cells of the heart, liver, muscle, pancreas or other organ or tissue cell. The pluripotent or multipotent AFS cells, when cultured under certain conditions, have the ability to differentiate in a regulated manner into three or more subphenotypes. Once sufficient cellular mass is achieved, cells can be differentiated into endodermal, mesodermal and ectodermal derived tissues in vitro and in vivo. This planned, specialized differentiation from undifferentiated cells towards a specific cell type or tissue type is termed “directed differentiation;” Exemplary cell types that may be prepared from pluripotent or multipotent AFS cells using directed differentiation include but are not limited to fat cells, cardiac muscle cells, epithelial cells, liver cells, brain cells, blood cells, neurons, glial cells, pancreatic cells, and the like.

### Nonidentical Donors—Sirs and Twins

Twins. The term “twinning” refers to two offspring developing with the same maternal parent and born at the same time. Twins can develop from the same zygote (monozygotic or “identical twins”) or from separate, zygotes (dizygotic or “fraternal twins”).

Dizygotic (DZ) or fraternal twins result when a woman double ovulates and each egg is independently fertilized. Genetically, DZ twins are as alike as ordinary siblings, sharing on average 50% of their genes.

In DZ twins, somatic cells originate from two products of conception. Therefore, their stem cells collected from DZ twins are derived from two products of conception. A transplant or graft made soley from a DZ collection is derived from two products of conception. A transplant or graft made from this collection is chimeric and will demonstrate the phenotype of two sibling genotypes. In the present invention, engraftability of transplant tissue relies on the ability of two different sources of stem cells obtained at the same or different times of ontogeny to competitively engraft and differentiate into multiple lineages in the same recipient (host) organism.

Nonidentical twins can be of the same sex (two brothers or two sisters) or of different sex; (brother and sister). Fraternal twins—the most common kind—occur when two separate eggs are fertilized by two different sperm. Each twin has his or her own placenta and amniotic sac. Dizygotic twins are dichorionic and diamniotic in placentation.

Monozygotic twins, in effect, are clones. They share 100% of their genes in common. Identical twins occur when a single fertilized egg splits and develops into two fetuses. Identical twins may share; a placenta, but each baby usually has a separate amniotic sac. Genetically, the two babies are identical.

Triplets and other higher order multiples can be identical, fraternal or a combination of both.

Twin zygosity is the genetic relationship between two twins. When two eggs are independently fertilized by two sperm cells, fraternal or dizygous twins result. A zygosity test is a comparison of the DNA between two twins. Amniocytes, chorionic villus, or blood samples from the twins can be used for analysis. The presence of different alleles between the twins is evidence of dizygosity.

Obtaining Amniotic Fluid Cells From Nonidentical Sibling Donors. For obtaining fluid from one or more amniotic sacs in a dizygotic pregnancy, a physician uses ultrasound images to guide needle placement. Subsequently, the fetal cells contained in the fluid are isolated and grown.

http://www.icongrouponline.com/Health/Amniocentesis_Ph.html “Haploidentical” refers to sharing a haplotype; having the same alleles at a set of closely linked genes on one chromosome.

Sibling donors (haploidentical siblings) include, stem cells taken from a brother or sister.

### Expansion and Differentiation

The goal of ex vivo expansion (of a culture or a coculture) is to increase the available dose of the cells responsible for successful engraftment, thereby reducing time to engraftment and reducing the risk of graft failure.

A variety of cell differentiation inducing agents can be used to differentiate cells in the polyclonal compositions of pluripotent fetal stem cells of the present invention into different phenotypes. To determine the differentiation status of the stem cells, the phenotypic characteristic of the cells are observed using conventional methods, such as light microscopy to detect cell morphology, RT-PCR to detect cell differentiation lineage specific transcription, and immunocytochemistry to detect cell surface markers specifically expressed in a particular cell's differentiation lineage. For example, genes expressed during the osteogenic differentiation serve as markers of the stem cells differentiating into osteogenic lineage (Long, Blood Cells Mol Dis 2001 May-June; 27(3):677-90).

Stem cell differentiation techniques are readily available for differentiating the compositions of pluripotent and multipotent AFS cells of this invention. These techniques are found in general texts such as: Teratocarcinomas and embryonic stem cells: A practical approach (E. J. Robertson, ed. IRL Press Ltd. 1987); Guide to Techniques in Mouse Development (P. M. Wasserman et al. eds, Academic Press 1993); Embryonic Stem Cell Differentiation in vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998); and in Stem cell biology (L. M. Reid, Curr. Opinion Cell Biol. 2:121, 1990), each of which is incorporated by reference herein in its entirety.

U.S. Published Application No. 2005/0124 003 provides detailed examples in which selection strategy specific marker positive cells were induced to differentiate into separate terminal cell lineages. The ability to induce specific differentiation was initially evident by morphological changes, and was confirmed by immunocytochemical and gene expression analyses. Generally, marker positive fetal stem cells can be differentiated into different cell lineages according to methods well known to one skilled in the art (Stem Cells: Scientific Progress and Future Research Directions. Appendix D; Department of Health and Human Services. June 2001. http: www.nih.gov news stemcell scireport.ht).

The stem cells of the polyclonal compositions of the invention can be selectively and specifically induced in culture, with various well known defined differentiation media referred to in the references herein, to differentiate into adipocytes, osteoblasts, skeletal muscle, endothelial cells, all of which express and display specific markers characteristic of their respective lineages.

The polyclonal, fetal stem cell compositions of the invention may also be expanded in the presence of an agent which suppresses cellular differentiation. Such agents are well-known in the art (DushnilcLevinson, M. et al., “Embryogenesis in vitro: Study of Differentiation of Embryonic Stem Cells,” Biol. Neonate, Vol. 67, 77-83, 1995). The cells may be assessed for viability, proliferation potential, and longevity using standard techniques in the art. Longevity may be determined by the maximum number of population doublings in extended cultures or other techniques known in the art.

U.S. Published Application No. 2005/0124003 discloses techniques which were; applied to monogenomic, in vitro cell cultures, each developed from amniotic fetal cells of an individual donor. These cultures were enriched for c-kit positive pluripotent fetal stem cells which are generally characterized in that; the cultures stain positive for c-kit and SSAE3 and SSAE4, produce progeny cells that can differentiate into at least two, preferably three, most preferably at least all of the following cell lineages: osteogenic, adipogenic, neurogenic, myogenic, hematopoietic, hepatic and endothelial cell lineages in the presence of differentiation-inducing conditions of which examples are described therein. Further examples of differentiation-inducing agents and combinations thereof for differentiating desired cell lineages can be found at Stem Cells: Scientific Progress and Future Research Directions. (Appendix D. Department of Health and Human Services. June 2001. http://www.nih.gov/news/stemcell/scireport.htm).

### Compositions of the Invention.

To form a composition of the present invention, a first monogenomic, in vitro cell culture (or portion thereof) of amniocytes is obtained from the amniotic fluid of an individual-donor. The first monogenomic culture is mixed with a second monogenomic, in vitro cell culture (or portion thereof) of amniocytes from the amniotic fluid of a second, genotypically different donor. Thus, a composition is formed, which comprises a combination of amniotic cells, or amniotic stem cells thereof, from at least two non-identical donors. In certain embodiments of this composition, the cells from one of the donors comprises a marker which distinguishes its cells from the cells of the other donor. As a non-limiting example, sex-linked markers, distinguish the cells of a male donor from a female donor.

Approximately equal proportions (i.e. 1:1) of cells of each genotype are used to make a mixed composition. Alternatively, the proportion of genotypes can be varied so long as the composition remains a mixed genotype composition.

Applicable to mixed genomic compositions of the invention, and operative in the present invention are techniques for differentiating amniotic fetal stem cells into (as non-limiting examples) neurogenic, osteogenic, adipogenic, myogenic and endothelial lineages (U.S. Published Application No. 2005/0124003).

### Banking & Cryopreservation

Applying the techniques of U.S. Published Application No. 2005 0042 595, polyclonal compositions of fetal stem cells are cryopreserved, i.e. frozen at liquid nitrogen temperatures and stored for long periods of time, thawed and reused. The cells will usually be stored in 5% DM50 and 95% fetal calf serum. Once thawed, the cells may be expanded by use of growth factors or stromal cells, associated with stem cell proliferation and differentiation.

The polyclonal compositions of cells may also be “banked” or stored in a manner that allows the cells to be revived as needed in the future, as disclosed in US Published Application No. U.S. 2005/0042595, incorporated by reference herein in its entirety. An aliquot of the undifferentiated cells can be removed at any time, to be differentiated into a particular cell type or tissue type, and may then be used to treat a disease or to replace malfunctioning tissues in a patient. Since the cells are harvested from the amniotic fluid, the cells can be stored.

The present invention also contemplates cryopreservation of the chorionic villus and amniotic fluid samples as well as the placenta samples, wherein once-thawed cells can be obtained. The amniotic fluid, chorionic villus, placenta tissue and fetal stem cells, before or after mixing or before or after differentiation, may be cryopreserved in a cryoprotective solution comprising a medium or buffer and a cryoprotective agent.

### Mixed Compositions of the Invention

An aspect of the invention resides in a composition which is a population of human fetal stem cells derived independently from non-identical donors, i.e. donors of at least two non-identical genotypes. The composition is a population of cells comprising at least a subpopulation of one genotype and a second subpopulation of another genotype.

In an embodiment of the composition, the mixed culture, for example, a cell suspension comprising sibling genotypes, is formed by mixing a population of amniotic fluid cells from one sibling and a population of amniotic fluid cells from the other sibling of a multigestational, dizygotic pregnancy. In one of its dimensions, the invention is a mixed composition comprising non-identical genotypes of amniotic fluid cells or stem cell derivatives thereof, each genotype obtained from separate non-identical donor fetuses. In certain embodiments, the mixed composition is a combination of two cell lines in a mixed culture formed from the cells of donors of at least two non-identical genotypes. An embodiment of the mixed composition comprises a mixture of amniotic fluid cells obtained from amniotic; fluids of non-identical fetuses of a twin gestation.

The mixed genomic compositions of the invention are formed from mixing monogenomic compositions of amniotic fluid cells or derivatives thereof obtained from separate donors of non-identical genotypes. It is understood that sibling genotype compositions are derived, as well, from siblings gestated at different times, a first, population of amniotic fluid cells obtained from the first gestation, cryopreserved and banked until amniotic fluid cells are obtained from gestation of the second sibling, whereupon the first and second populations, in their entirety or aliquots thereof, are mixed. Unused portions of the first and/or second cryopreserved populations may be kept cryopreserved for future use.

Another dimension of the invention is directed to a tissue transplant comprising a composition of the invention. Essentially, the tissue transplant-comprises amniotic cells which are allogeneic to each other.

A further dimension of the invention is directed to a method of regenerating tissue in a recipient by transplanting a population of cells which comprises a mixed genotype composition of the invention into that recipient. Cells of the transplant colonize and engraft structurally and functionally into the recipient tissue.

An aspect of the invention is directed to methods of culturing pure populations of human amniotic stem cells of one genotype with at least amniotic stem cells of another genotype.

A further aspect involves methods of fabricating a transplant which involves a step of combining amniotic fluid cells of one genotype with at least amniotic fluid cells of another genotype.?

The phenotype of a culture of a chimera of the invention is the phenotype of at least two genotypes. For example, the phenotype of a mixed culture is the product of the phenotype of one subpopulation, plus the phenotype of the other subpopulation in the population of the mixed composition.

In a method of the invention, chimeric transplants are formed from coculturing and differentiating two genotypes of amniotic fluid cells Approximately equal proportions (i.e. 1:1) of cells of each genotype are used to make a mixed composition. Alternatively, the proportion of genotypes can be varied so long as the composition remains a mixed genotype composition.

It should be understood that preparation and maintenance of the mixed compositions of the invention rely on methods well known in the art for obtaining, culturing, expanding/enriching/selecting, and transplanting monogenomic preparations of amniotic fluid cells or stem cell derivatives thereof. Tissue culture techniques well known in the art allow practitioners to culture pure populations of donor cells of one genotype with another donor's cells of another genotype under conditions that permit expansion and/or differentiation of mixed genotype culture's and the preparation of transplants derived therefrom. The written description and enablement of isolating, characterizing, selecting and differentiating stem cells from genotypically different human embryonic and fetal chorionic villi and amniotic fluid are set forth in U.S. Published Application No. 2005/0124003. Specific marker positive amniotic fluid or stem cells from early to late passages, were inducible to different cell types including osteogenic, adipogenic, myogenic, neurogenic and endothelial cell lineages under specific growth factors. The ability to induce specific differentiation was initially evident by morphological changes, and was also confirmed immunocytochemically, by gene expression patterns and cell-specific functional analyses.

The chimeric cultures and transplants of the invention have genetic expression profiles and immunophenotypes which demonstrate the effects of genetic heterogeneity obtained from amniotic fluid cells, and stem cell derivatives thereof, derived from different donors.

Accordingly, two amniotic cell populations with distinct expression profiles can be obtained from different donors, and cultured separately. From each of these cell populations (for the sake of illustration, referred to now as Type 1 and Type 2), clones or cell lines; may be isolated as individual colonics. The two types bear phenotypic differences as Type 1 and Type 2 amniotic fluid cells MSCs are from genetically different sources. Type 1 and Type 2 cell lines have unique molecular signatures for each type, as measured through, for example, gene expression studies on each cell population.

Certain embodiments of the invention involve mixed, stem-cell enriched compositions. These compositions are formed by mixing genotypically different amniotic cell lines or clonal lines.

As described below, methods of stem cell transplantation are based on transplanting mixed genotype compositions (i.e. chimeras) of the invention. The therapeutic utility of the transplant cell biology of the invention is based upon a mixed composition of pluripotent stem cells from the amniotic sacs of non-identical individuals or non-identical siblings. Engraftment of the mixed genotype transplants of the invention involves, as with monoclonal transplants, movement of the transplanted cells to specific-microenvironmental “niches” that favor self-renewal and differentiation of a mix of donor cells of two genotypes.

The present invention relies oh amniotic fluid as an abundant source of fetal amniotic stem cells. Monogenomic populations of cells are derived from independently-harvested amniotic fluid samples which are obtained from genotypically different donors. Combined, the independently harvested cell samples exhibit multilineage differentiation potential as they would have had they not been combined.

It is well known in the art that monogenomic compositions of amniotic fluid cells (or chorionic villi) represent a viable source of human fetal stem cells that can be used therapeutically. Monogenomic compositions of amniotic fetal stem cells, that maintain both their pluripotential and proliferative ability, have been identified and isolated. The present invention relies upon use of such monogenomic composition form a mixed composition that comprises at least two genotypes of amniotic fluid cells or the stem cells derived therefrom.

### Competitive Repopulation Assays

Preferred embodiments of the invention involve mixed compositions in which the cells of one of the donors has a distinguishable phenotypic marker (as expressed, for example, by selectable cell surface marker genes) in comparison to the cells of the other donor.

After ex vivo expansion, the engraftability of the mixed stem cell compositions of the invention relies on a first donor's (Donor 1 haying genotype 1) amniotic stem cell regenerative power in mixture with a second donor having a second genotype (Donor 2 having genotype 2) which has its respective amniotic cell regenerative power to reconstitute target tissue of a host.

Competitive repopulation assays measure the relative ability of each donor cell population to reconstitute stem cell-depleted recipients (Rosier ES et al. An in vivo competitive repopulation assay for various sources of human hematopoietic stem cells. Blood. 2000 Nov. 15; 96(10):3414-21).

The contribution of each genotype of cell in a chimeric graft of the invention is assayed by competitive repopulation assays. Competitive repopulation assays are based on a principle of quantifying stem cell numbers or assessing stem cell function through competition with stem cells of known number or function. Normal amniotic fluid cells from one individual compete roughly equally against normal amniotic fluid cells from genotypically different second individual. Stem cell function in each donor of a particular genotype is assayed by mixing its amniotic fluid cells with a number of like cells from a donor with distinguishable markers), and measuring the relative ability of each donor to populate stem cell-depleted recipients. Model systems are well known in the art for determining the relative contributions of each donor genotype to the repopulating capacity of the mixed graft. In addition, a variety of in situ cell markers have been developed to allow tracing of the distribution of the contributing cell types. The inclusion of independent genetic markers to follow the fate of the two cell components is critical.

### Transplant Therapeutics: Treating Individuals in Need of a Transplant

The isolated mixed composition of amniotic cells, or their derivatives, may be used to treat diseases in humans and animals. As used herein the terms “treat” or “treatment” refer to both therapeutic treatment and prophylactic or preventative measures, wherein the object is to prevent, slow down (lessen), or reverse an undesired physiological change or disorder. The term “treat” also refers to the characterization of the type or severity of disease which may have ramifications for future prognosis, or need for specific treatments. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival in the absence of treatment. Those in need of treatment include those already with the condition or disorder, those prone to have the condition or disorder, and those in which the condition or disorder is to be prevented.

To treat a human or animal in need of treatment, the chimeric transplants of the invention are either regenerated into segments of a desired tissue, and transplanted into the patient, regenerated into a whole tissue used to replace the failing tissue, or injected into a tissue of interest as whole cells, where the transplanted cells regenerate at the injected location. Chiavegato A, et al. Human amniotic fluid-derived stem cells are rejected after transplantation in the myocardium of normal, ischemic, immunosuppressed or immuno-deficient rat.; J Mol Cell Cardiol. 2007 April; 42(4):746-59; De Filippo, Roger, Grant No. 1K08DK073082-02, “Renal Tissue Derived From Amniotic Fluid Stem Cells,” (National Institute of Diabetes and Digestive and Kidney Diseases); Delo, Dawn M, Grant No. 1F31AA016056-01, “Stem Cells-Treatment for alcohol-Related Heart Disease,” (National Institute on Alcohol Abuse and Alcoholism); Soker, Shay, Grant No. 1R41DK077256-01, “Cell Therapy of diabetes using broad spectrum multipotent cells, (National Institute of Diabetes and digestive and Kidney Diseases); U.S. Published Application No. 20070031384, Regeneration of Pancreatic Islets by Amniotic Fluid Stem Cell Therapy.

The invention contemplates replacing any type of failing tissue in an individual with mixed compositions of the invention. The mixed composition of amniotic cells may be differentiated into tissues such as liver, endocrine tissues, lung, blood cells, neuronal or astroglial cells, or others, which may then be used for transplantation to cure or treat diseases. Examples of diseases that may be treated with amniotic fluid-derived cells or tissues include but are not limited to cirrhosis of the liver, pancreatitis, diabetes, Parkinson's disease, spinal cord injury, stroke, burns, heart disease* certain types of cancer, osteoarthritis, rheumatoid arthritis, leukemia, lymphoma, genetic blood disorders, and brain disorders such as Alzheimer's disease.

In transplant therapeutics, a genetically monoclonal graft is one in which all of the cells are derived from a single donor, which is to say, all of the cells in the graft are the same genotype. Under such circumstances, transplant therapeutics is a monoclonal practice, in which clonal (i.e. monoclonal) donor cells are transplanted to a clonal host. In monoclonality, the graft composition reflects the proliferation, self-renewal and multipotency of a single founding cell. Host (recipient) cells all derive from the same cell (i.e. a zygote), and therefore all the cells in one recipient patient will have the same genotype. In the instance of a bi-clonal graft, the host tissue responds to bi-clonality in reconstituted tissue.

Additional examples of diseases that can be treated with mixed amniotic fluid-derived cells include but are not limited to Acute Lymphoblastic Leukemia, Acute Myelogenous Leukemia, Acute Biphenotypic Leukemia, and Acute Undifferentiated Leukemia; Chronic Myelogenous Leukemia, Chronic Lymphocytic Leukemia, Juvenile Chronic Myelogenous Leukemia, Juvenile Myelomonocytic Leukemia, Refractory Anemia, Refractory Anemia with Ringed Sideroblasts, Refractory Anemia with Excess Blasts, Refractory Anemia with Excess Blasts in Transformation, Chrunic Myelumunucytic Leukemia, Aplastic Anemia, Fanconi Anemia, Paroxysmal Nocturnal Hemoglobinuria, Pure Red Cell Aplasia, Acute Myelofibrosis, Agnogenic Myeloid Metaplasia, myelofibrosis, Polycythemia Vera, Essential Thrombocythemia, Non-Hodgkin's Lymphoma, Hodgkin's Disease, Chediak-Higashi Syndrome, Chronic Granulomatous Disease, Neutrophil Actin Deficiency, Reticular Dysgenesis, Mucopolysaccharidoses, Hurler's Syndrome, Scheie Syndrome, Hunter's Syndrome, Sanfilippo Syndrome, Morquio Syndrome, Maroteaux-Lamy Syndrome, Sly Syndrome, Beta-Glucuronidase Deficiency, Adrenoleukodystrophy, Mucolipidosis II, Krabbe Disease, Gaucher's Disease, Niemann-Pick Disease, Wolman Disease, Metachromatic Leukodystrophy, Familial Erythrophagocytic Lymphohistiocytosis, Histiocytosis-X, Hemophagocytosis, Inherited Erythrocyte Abnormalities, Beta Thalassemia Major, Sickle Cell Disease, Inherited Immune System Disorders, Ataxia-Telangiectasia, Kostmann Syndrome, Leukocyte Adhesion Deficiency, DiGeorge Syndrome, Bare Lymphocyte Syndrome, Omenn's Syndrome, Severe Combined Immunodeficiency, Common Variable. Immunodeficiency, Wiskott-Aldrich Syndrome, X-Linked Lymphoproliferative Disorder, Other Inherited Disorders, Lesch-Nyhan Syndrome, Cartilage-Hair Hypoplasia, Glanzmann Thrombasthenia, Osteopetrosis, Inherited Platelet Abnormalities, Amegakaryocytosis, Congenital Thrombocytopenia, Plasma Cell Disorders, Multiple Myeloma, Plasma Cell Leukemia, Waldenstrom's Macroglobulinemia, Breast Cancer, Ewing Sarcoma, Neuroblastoma, Renal Cell Carcinoma, brain disorders such as Alzheimer's disease, and the like (see, for example, hypertext transfer protocol (http) on the world wide web at: marrow.org/index.html, which is incorporated by reference herein in its entirety).

Many different types of tissues may be partially or completely replaced using the differentiated cells derived from the mixed composition of amniotic fluid cells, as described herein. Examples of tissues which may be (at least partially) replaced include, but are not limited to, lung tissue, heart tissue, ocular tissue, nerve tissue, brain tissue, muscle tissue, skin, pancreatic beta cells, and the like.

The use of substantially purified or enriched marker positive pluripotent fetal stem cells of the present invention are useful in a variety of ways. In a polyclonal composition of the invention, an enriched mixed composition of amniotic stem cells is used to reconstitute a host whose cells have been lost through disease or injury. Genetic diseases associated with cells may be treated by genetic modification of stem cells in polyclonal compositions to correct a genetic defect or treat to protect against disease.

Alternatively, polyclonal compositions of normal allogeneic fetal stem cells may be transplanted. Diseases other, than those, associated with cells may also be treated, where the disease is related to the lack, of a particular secreted product such as hormone, enzyme, growth factor, or the like. CNS disorders encompass numerous afflictions such as neurodegenerative diseases (e.g. Alzheimer's and Parkinson's), acute brain injury (e.g. stroke, head injury, cerebral palsy) and a large number of CNS dysfunctions (e.g. depression, epilepsy, and schizophrenia). In recent years, neurodegenerative disease has become an important concern due to the expanding elderly population which is at greatest risk for these disorders. These diseases, which include Alzheimer's Disease, Multiple Sclerosis (MS), Huntington's Disease, Amyotrophic Lateral Sclerosis, and Parkinson's Disease, have been linked to the degeneration of neural cells in particular locations of the CNS leading to the inability of these cells or the brain region to carry out their intended function.

By providing for maturation, proliferation and differentiation into one of more selected differentiation lineages through specific different growth factors, the polyclonal composition of progenitor cells, may be used as a source of committed cells. The polyclonal composition of pluripotent fetal stem cells according to the present invention can also be used to produce a variety of blood cell-types, including myeloid and lymphoid cells, as well as early hematopoietic cells (see, Bjornson et al., 283 Science 534 (1999), incorporated herein by reference).

Compositions, of polyclonal fetal stem cells according to the present invention can also be used as a tool for the repair of a number of CNS disorders as described in a review by Cao et al. (Stem cell repair of central nervous system injury, J. Neuroscience Res. 68:501-510, 2002) which is incorporated by reference.

The polyclonal composition of cells may be administered to a patient as a method of gene therapy by administering genetically modified multipotent or pluripotent cells of the invention.

In summary, polyclonal stem cell, compositions of the present invention are formed by mixing two or more such populations, each obtained from a donor of a different genotype. If the donor genotypes are siblings, then the transplant is essentially a co-transplanted sibling haploidentical composition of amniotic fluid cells or derivatives thereof. Embodiments of the invention include a polyclonal composition having multipotent or pluripotent stem cells derived from amniotic, fluid donors of different genotypes.

The amniotic fluid cells of the mixed compositions may be undifferentiated, or they may also be differentiated cells derived from the above-described multipotent or pluripotent stem cells.

The present invention provides methods of preparing polyclonal compositions of multipotent or pluripotent stem cells, by isolating the stem cells from, amniotic fluid of donors of different genotypes, purifying the stem cells, and growing the stem cells in or on a medium, during which a preparation the cells of one genotype are mixed with the cells of the other genotype.

### Assays

The polyclonal composition of selected fetal stem cells may also be used in the isolation and evaluation of factors associated with the differentiation and maturation of cells. Thus, the mixed genotype composition of cells serve as a model or “tissue equivalent” intended to mimic the physiology of cells or tissues in situ, and used in assays to determine the activity of media, such as conditioned media, evaluate fluids for growth factor activity, involvement with dedication of lineages; or the like.

Certain embodiments of the polyclonal compositions of the invention relate to an in vitro, ex vivo or in vivo assay. Accordingly, a mixed composition is used for determining the biological activity of pharmaceutical and/or biological agents. This utility generally involves contacting the cells in the mixed composition with a test agent, and determining the biological activity the test agent has on at least one genotype of cells seeded into the composition. The test agent may be administered to a mixed composition in vitro, or it may be administered to the mixed composition before and/or after the mixed composition is transplanted into a recipient. In the environments noted, the biological effects of the test agent on the seeded cells or cells that engraft in the recipient may be measured. Biological effects measured with the inventive mixed compositions include, but are not limited to cytotoxicity, mutagenicity, proliferation, permeability, apoptosis, cell-to-cell interactions, gene regulation, protein expression, cell differentiation, cell migration and tissue formation. Test agents may be assessed, individually, or as a combination of test agents.

The biological activity of a test agent on a tissue equivalent may be measured using a variety of techniques known in the art. Cytoxicity, for example, may be measured using surrogate markers including, but not limited to, neutral red uptake, and lactate dehydrogenase release, and malondialdehyde levels (see e.g. Zhu et al.

“Cytotoxicity of trichloroethylene and perchloroethylene on normal human epidermal keratinocytes and protective role of vitamin E” Toxicology April 1; 209(1):55-67 Epub 2005 Jan. 7; and U.S. Pat. No. 5,891,161; these disclosures are incorporated herein by reference). Cytoxicity may also be measured by microscopically comparing the numbers of live cells before and after the mixed composition is exposed to a test agent.

Cytotoxicity may be measured by detecting the metabolic reduction of a soluble tetrazolium salt to a blue formazan precipitate since this reaction is dependent on the presence of viable cells with intact mitochondrial function. This assay is used to quantitate cytotoxicity in a variety of cell types (see e.g. U.S. Pat. No. 5,891,617 A, incorporated herein by reference). Other methods for measuring cytoxicity include examination of morphology, the expression or release of certain markers, receptors or enzymes, on DNA synthesis or repair, the measured release of [3H]-thymidine, the incorporation of BrdU, the exchange of sister chromatids as determined by metaphase spread (see U.S. Pat. No. 7,041,438 B2 and “In vitro Methods in Pharmaceutical Research”, Academic Press, 1997; these are incorporated herein by reference), and the differential incorporation of specific dyes by viable and non-viable cells (see e.g. U.S. Pat. No. 6,529,835 B1, incorporated herein by reference). Methods for analyzing an effect of a candidate compound on cellular coculture are disclosed in U.S. Patent Publication No. 2003 0215 941, incorporated by reference.

The mixed compositions also provides methods of screening for agents that promote, inhibit or otherwise modulate the differentiation and/or proliferation of stem cells. There are a number of proliferation and differentiation assays known in the art including those disclosed in U.S. Pat. Nos. 7,037,719, 6,962,698, 6,884,589 and 6,824,973, the disclosures of which are incorporated herein by reference. In general, these assays involve culturing a population of progenitor cells in the presence of a test agent, and monitoring the proliferative and/or differentiating effects that the test agent imparts on the progenitor cell population. One skilled in the art will appreciate that there are a number of methods for monitoring these effects including, but not limited to, testing for the presence of lineage-identifying cell surface markers, microscopic analysis of cell morphology, histological examination of extracellular proliferation markers, and cell counts.

The effects of test agents may also be evaluated using histochemical, immunohistochemical, and immunofluorescent methods to establish the presence of absence of specific proteins, glycoproteins, and proteoglycans (Results measured by histology, histomorphometry and immunochemistry (Br. J. Dermatol. (2004) 151, (4):753-65). According to this embodiment, the tissue equivalent is exposed to a test agent and subsequently frozen, embedded in a suitable embedding composition and sectioned for determination of cellular or extracellular enzymatic activities, and peptide and protein functionality. Alternatively, the tissue equivalents may be fixed, embedded in paraffin or other suitable embedding composition and sectioned for examination using optical microscopy. Tissue equivalent can also be used to assess gene expression by in situ hybridization with nucleotide probes complementary to specific nucleic acid sequences.

### Genetic Modifications

Additional embodiments include polyclonal compositions of multipotent or pluripotent cell derived from amniotic fluid which cells have been genetically modified. For example, the cell may be genetically modified to express TERT. Further embodiments of the invention include methods of gene therapy treatment of a patient, by administering genetically modified multipotent or pluripotent cells.

The amniotic fluid cells or derivatives thereof may also be genetically modified by transfection with any suitable gene of interest. General techniques useful to genetically modify the cells (or their derivatives) can be found for example, in standard textbooks and reviews in cell biology, tissue culture, and embryology. Methods in molecular genetics and genetic, engineering are described, for example, in Molecular Cloning: A Laboratory Manual, 2nd Ed. (Sambrook et al., 1989); Oligonucleotide Synthesis (M. J. Gait, ed., 1984); Animal Cell Culture (R. I. Freshney, ed., 1987); the series Methods in Enzymology (Academic Press, Inc.); Gene Transfer Vectors for Mammalian Cells (I. M. Miller & M. P. Cabs, eds., 1987) Current Protocols in Molecular Biology and Short Protocols in Molecular Biology, 3rd Edition (F. M. Ausubel et al., eds., 1987 & 1995); and Recombinant DNA Methodology II (R. Wu ed., Academic Press 1995); each of which is incorporated by reference herein in its entirety.

The mixed cell compositions of the invention may be isolated in a similar manner from other species. Examples of species that may be used to derive the inventive compositions include but are not limited to, mammals, humans, primates, dogs, cats, goats, elephants, endangered species, cattle, horses, pigs, mice, rabbits, and the like.

The references cited herein and throughout the specification are incorporated by reference in their entirety.

### BIBLIOGRAPHY

De Coppi, P. et al., Isolation of Amniotic Stem Cell Lines With Potential for Therapy, 2007, Nature Biotechnology 25(1): 100-106.
U.S. Published Patent Applications: 2005-124 003 (Atala); 2005.0054 09.3 (Haas) and 2005 0042 595 (Haas);
(Bossolasco P et al.) Molecular and phenotypic characterization of human amniotic fluid cells and their differentiation potential Cell Research (2006)16:329-336
Elen S. Rosier et al., (Blood. 2000; 96:3414-3421) An in vivo competitive repopulation assay for various sources of human, hematopoietic stem cells
J. Kim, et al., Human amniotic fluid-derived stem cells have characteristics of multipotent stem cells; Cell Prolif 2007, 40: 75-90
Sakuragawa N, Thangavel R, Mizuguchi M, Hirasawa M, Kamo I. Expression of markers for both neuronal and glial cells in human amniotic epithelial cells. Neurosci Lett 1996; 209: 9-12.
Sakauragawa et al. US 2003 0044977 Human Stem Cells Originated From Human Amniotic Mesenchymal Cell Layer; International Journal Of Molecular Medicine 16: 987-992, 2005
Karlin R. Karlmark et al., Activation of ectopic Oct-4 and Rex-1 promoters in human amniotic fluid cells. International Journal Of Molecular Medicine 16: 987-992, 2005
Jane S. Reese et al, Repopulating Defect of Mismatch Repair-Deficient Hematopoietic Stem Cells, Blood First Edition Paper, prepublished online May 1, 2003; DDI 10.1182/blood-2002-10-3035, Circulation. 2005; 112:1618-1627; published Online Sep. 6, 2005;
Chang-Hwan Yoon, et al, Synergistic Neovascularization by Mixed Transplantation of Early Endothelial Progenitor Cells and Late Outgrowth Endothelial Cells; The Role of Angiogenic Cytokines and Matrix Metalloproteinases, (Circulation 2005; 112:1618.)
Ming-Song Tsail et al, Isolation of human multipotent mesenchymal stem cells from second-trimester amniotic fluid using a novel two-stage culture protocol; Human Reproduction, Vol. 19, No. 6, 1450-1456, June 2004
Ming-Song Tsai, et al, Clonal Amniotic Fluid-Derived Stem Cells Express Characteristics of Both Mesenchymal and Neural Stem Cells, Human Reproductions Vol. 19, No. 6, 1450-1456, June 2004
Two-stage culture protocol for isolating mesenchymal stem cells from amniotic fluid; Patent Inventors Ming-Song Tsai, Shiaw-min Hwang; US Published Patent Application No. 20050118712 http://www.icongrouponoline.com/Health/Amniocentesis_Ph.html Enhancement of amniotic fluid cell growth in culture; Stem Cells Dev. 2004 October; 13(5):548-62.

Maintenance and self-renewal of long-term reconstituting hematopoietic stem cells supported by amniotic fluid. Barria E, Mikels A, Haas M., Department of Biology/Cancer Center, University of California, San Diego, La Jolla, Calif. 92093-0063, USA.
Prusa A-R et al., “Oct-4-expressing cells in human amniotic fluid: a new source for stem cell research?,” Human Reproduction 18; 1489-1493, 2003.
De Gemmis, PD et al. A Real-Time PCR Approach to Evaluate Adipogenic Potential of Amniotic Fluid-Derived Human Mesenchymal Stem Cells, Stem Cells Dev. 2006 October; 15(5):719-28.
Pieternella S. in 't Anker, Sicco A. Seherjon, Carin Kleijburg-van der Keur, Willy A. Noort, Frans H. J. Claas, Roelof Willemze, Willem E. Fibbe, and Humphrey H. H. Kanhai (2003) Blood, 15:1548-1549.
U.S. Pat. No. 6,790,614; Pippig, et al. Sep. 14, 2004
Fauza D, Amniotic fluid and placental stem cells, Best Pract Res Clin Obstet Gynaecol. 2004 December; 18(6):877-91.
DeCoppi et al. Nature 25: (1): 100-106
U.S. Published Patent Applications: 2005-124 003 (Atala); 2005 0054 0.93 (Haas) and 2005 0042 595 (Haas)

What is claimed is:

1. A composition comprising amniotic fluid stem cells or derivatives thereof derived from at least two non-identical donors.

2. The composition of claim 1 wherein the cells from one of said donors comprise a phenotypic marker which distinguishes its cells from the cells of another of said donors.

3. The composition of claim 1 wherein said non-identical donors are non-siblings.

4. The composition of claim 2 wherein said donors are siblings.

5. The composition of claim 3 wherein said siblings are non-identical twins.

6. The composition of claim 1 having a phenotype derived from sibling genotypes.

7. A method of creating a chimeric composition comprising amniotic fluid cells or derivatives thereof derived from at least two non-identical donors, comprising the steps of:
(a) obtaining a first culture of amniotic fluid cells of one genotype from a first donor;
(b) obtaining a second culture of amniotic fluid cells of another genotype from another donor; and
(c) co-culturing said first culture and said second culture such that a chimeric composition is formed.

8. A chimeric transplant comprising a composition which comprises amniotic stem cells derived from at least two non-identical donors.

9. A method of treating an individual in need of a transplant, said method comprising the step of administering to said individual a chimeric transplant which comprises a composition comprising amniotic stem cells derived from at least two non-identical donors.

10. A method of forming a chimeric transplant, comprising the steps of
(a) obtaining a culture of amniotic fluid cells of one genotype from a first donor;
(b) obtaining a second culture of amniotic fluid cells of another genotype from another donor; and
(c) co-culturing (a) and (b) such that a chimeric composition is formed.

11. A stem cell composition suitable for administration to a human recipient, comprising a therapeutically effective combination of stem cells derived from at least two nonidentical donors.

12. The composition of claim 11 wherein administration of said therapeutically effective combination induces regeneration of tissue in said recipient.

13. A method of preparing a cultured cell population containing amniotic fluid cells from at least a first and second human donor genotypically different from each other for administration to a human patient, comprising the steps of:
a) coculturing ex vivo human amniotic fluid cells from at least said first, and second human donors;
b) harvesting the cocultured cells; and
c) preparing the harvested cells for human administration.

14. A method of transplanting a mixed population of amniotic fluid cells or stem cells derived therefrom, comprising administering an expanded population of said mixed population to a patient in need thereof.

15. The method of claim 14, wherein said expanded population is; administered in ah amount effective to promote restoration or repair of tissue in said patient.

16. The method of claim 14, wherein said mixed population is transplanted in an amount effective to provide engraftment of at least a portion of said mixed population in said patient.

17. A competitive repopulation assay for measuring the relative contribution of each genotype of donor cell in a mixed genotype transplant formed from an amniotic stem cell composition, wherein the cells of one of the donors has a distinguishable phenotypic marker in comparison to the cells of at least one of the other donors, said assay comprising the steps of:
a. transplanting said transplant to a recipient; and
b. tracing the distribution of said distinguishable marker in one or more tissues of said recipient; and
c. quantitating the donor cell types in said one of more tissues thereby determining the relative ability of each donor to populate said one or more tissues of said recipient.

18. A method of analyzing an effect of candidate compound on, a cellular coculture comprising
a. forming a coculture ex vivo of human amniotic fluid cells or derivatives thereof from at least said first and second human donors;
b. contacting said coculture with a dose of at least one test compound;
c. performing an assay on the cells contacted in step (b).
